(19) United States
(12) Patent Application Publication
(10) Pub. No.: US 2026/0033891 A1
(43) Pub. Date: Feb. 5, 2026

(54) Tool and Method for Cardiac Ablation Using Hydrogel

(71) Applicant: Texas Heart Institute, Houston, TX (US)

(72) Inventors: Allison D. POST, Houston, TX (US); Mathews M. JOHN, Houston, TX (US); Skylar J. BUCHAN, Houston, TX (US); Drew BERNARD, Houston, TX (US); Mehdi RAZAVI, Houston, TX (US)

(21) Appl. No.: 19/099,446

(22) PCT Filed: Jul. 20, 2023
(86) PCT No.: PCT/US2023/028278

§ 371 Date: Jan. 28, 2025

Related U.S. Application Data
(60) Provisional application No. 63/390,920, filed on Jul. 20, 2022.

(51) Int. Cl. A61B 18/14; A61B 18/00; A61B 18/12

(52) U.S. Cl. CPC A61B 18/1492; A61B 18/1206; A61B 2018/00065; A61B 2018/00077; A61B 2018/00136; A61B 2018/00178; A61B 2018/00351; A61B 2018/00577; A61B 2018/126; A61B 2018/1465; A61B 2018/1475

(57) ABSTRACT

A catheter deploys a flexible electrode that is at least partially made of preformed hydrogel to a target location in order to provide radiofrequency catheter ablation. The catheter stores the flexible electrode, which can be deployed and retracted. The preformed hydrogel is conductive and thus can provide an interface between a conductive metal wire and a target cardiac tissue. Electric energy is applied to the portions of the cardiac tissue target tissue that are the genesis of the errant and abnormal heartbeats via the flexible electrode. The electric energy may create lesions with lower risks of steam pops and tissue perforation.

### CROSS-REFERENCE TO RELATED APPLICATIONS

This application claims the benefit of priority to U.S. provisional application Ser. No. 63/390,920, filed on Jul. 20, 2022, which is incorporated herein by reference.

### FIELD OF THE INVENTION

This disclosure relates generally to the utilization of hydrogels for forming flexible electrodes that can be placed in contact with a target tissue to safely create ablation lesions. This disclosure relates more particularly to a tool configured to deploy a wide, conductive hydrogel electrode that can generate wide, deep, and contiguous lesions and a method of ablation using the said tool. While the disclosure primarily discusses the ablation of cardiac tissue, potential uses of the tool also include the ablation of nerves or other tissues, such as liver or kidney tissues.

### BACKGROUND OF THE INVENTION

Cardiac arrhythmias, or abnormal rhythm, can be treated in several ways, including cardiac ablation, a procedure for destroying the tissue that is the genesis of the errant, and abnormal heartbeats. The most common form of ablation is radiofrequency (RF) ablation. Radiofrequency catheter ablation (RFCA) focuses on safely applying radiofrequency electric energy to the target tissue to form lesions. However, RFCA from traditional metal catheter tips is associated with complications such as steam pops, tissue perforation, or tissue charring. Steam pops are air pockets that can violently rupture, potentially bursting through the heart wall and causing massive internal bleeding. These air pockets can also travel to the lungs or brain, causing pulmonary embolism or a stroke.

For example, while the uniformity of lesions is dictated by the biophysics of ablation, the width of the lesions formed is limited by the size of the electrode in contact with the tissue. It is usually not possible to deploy large metal electrodes safely into the myocardium due to risks such as steam pops. Thus, RF ablation is limited in its traditional delivery by small metallic electrodes.

Moreover, in order to achieve transmural, or full tissue thickness, lesions, electric energy could be applied at high outputs (high power) for long periods of time. However, these high outputs and/or long periods of time can possibly result again in steam pop formation. Thus, currently, safety concerns limit the amount of RF energy that can be delivered to create wide lesions.

Thus, there is a need to address safety concerns, such as steam pops and tissue perforation, arising from RFCA with traditional metal catheter tips.

### SUMMARY

In some aspects, this disclosure describes an apparatus for ablating tissue. The apparatus may comprise a catheter having a proximal end adapted for a user and a distal end adapted for introduction in a patient. A flexible electrode may be located at the distal end, the flexible electrode including a cured hydrogel. The flexible electrode may comprise sensors.

In some aspects, this disclosure describes a system including the apparatus for ablating tissue. The system may comprise an electric generator connected to the flexible electrode and sensor readers.

In some aspects, this disclosure describes a method of ablating tissue. The method may comprise the steps of providing the system, introducing the catheter in a patient, contacting a target tissue of the patient with the flexible electrode, and ablating the target tissue. In a preferred embodiment, the target tissue is cardiac tissue, which is the genesis of the errant and abnormal heartbeats.

### BRIEF DESCRIPTION OF THE DRAWINGS

For a more detailed description of the embodiments of the disclosure, reference will now be made to the accompanying drawings, wherein:

FIG. 1 is a perspective view illustrating a catheter with a flexible electrode that is at least partially made of preformed hydrogel, retracted inside the catheter;

FIG. 2 is a perspective view illustrating the catheter shown in FIG. 1 with the flexible electrode deployed against a target tissue;

FIG. 3 is a sectional view illustrating a portion of the catheter shown in FIG. 1; and

FIG. 4 is a side view of the portion shown in FIG. 3.

### DETAILED DESCRIPTION

This disclosure describes a catheter that deploys a flexible electrode that is at least partially made of preformed hydrogel to a target location in order to provide catheter ablation, preferably radiofrequency catheter ablation. For example, the catheter stores the flexible electrode, which can be deployed and retracted. The preformed hydrogel, preferably conductive, provides an interface between a solid electrode (e.g., a conductive metal wire) and a target tissue. Electric energy, preferably at radiofrequency, provides ablation, such as by heating the target tissue.

Initial preliminary testing indicates that hydrogels can deliver wide lesions that are uniform and reduce the risk of steam pop by evenly distributing heat and even trapping air.

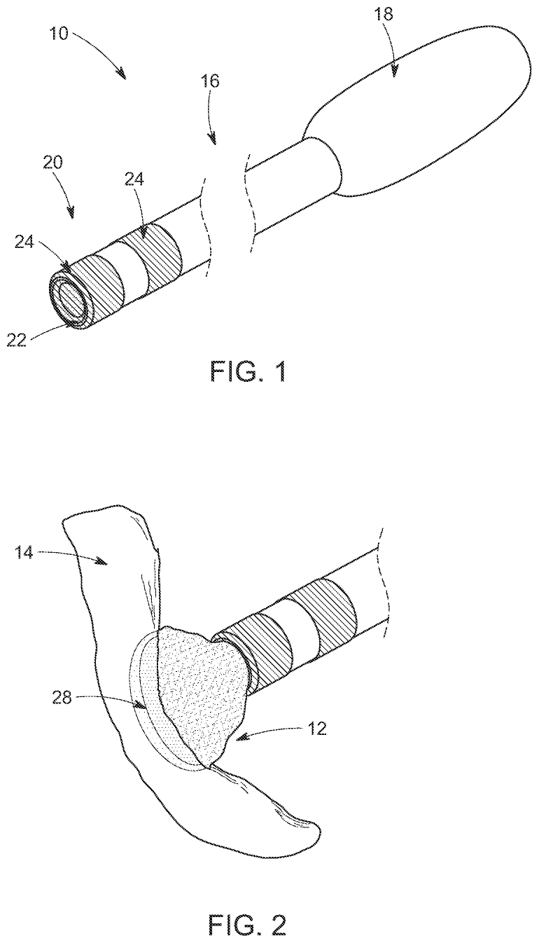

In reference to FIGS. 1 and 2, a preferred embodiment of ablation system is illustrated. This ablation system is designed to deliver RF energy to ablate cardiac tissue. Hydrogel delivery of RF energy appears to mitigate safety concerns, such as steam pops and tissue perforation, while allowing the creation of wide, continuous lesions. In contrast, these safety concerns currently limit the spreading and amount of RF energy that can be delivered to create wide lesions.

This ablation system comprises an electric generator (not shown), preferably a pulsed radiofrequency (RF) generator capable of repeatedly generating voltage wave trains at a frequency between 0.1 and 1 MHz for a duration between 1 millisecond and 100 milliseconds.

The ablation system comprises a steerable catheter 10 coupled to the generator. The steerable catheter 10 can deliver RF ablation through a flexible electrode 12, which is illustrated retracted in the catheter 10 in FIG. 1 and deployed against target tissue 14 in FIG. 2. The flexible electrode 12 is large compared to a needle and includes one or more preformed hydrogels. The flexible electrode 12 provides a flexible tip for ablating the target tissue 14. The flexible electrode 12 can be used for creating wide, uniform lesions 28 in the target tissue 14. For example, lesions that take on the shape of the contact area of the flexible electrode 12 have been made. Therefore, the larger the contact area between the flexible electrode 12 and the target tissue 14, the larger the lesion 28. The flexible electrode 12 can exhibit protective characteristics during RF ablation, such as steam pop prevention and even heat distribution across the contact area of the flexible electrode 12 with the target tissue 14. The steerable catheter 10 comprises a body 16, a proximal end 18, and a distal tip 20.

In the preferred embodiment, the distal tip 20 can comprise one or more electrodes 24 placed outside a lumen 22 of the catheter body 16. The distal tip 20 houses at least one distal solid electrode and always maintains contact with the preformed hydrogel. The one or more electrodes 24 on the lumen 22 of the catheter body 16 are used as sensing electrodes to locate the position of the flexible electrode 12 relative to the target tissue 14. The distal tip 20 can be provided with a plurality of ports for irrigation (e.g., with a cooling fluid).

The proximal end 18 of the catheter 10 includes the proximal handle or hub with which the user interacts. The proximal handle or hub thus controls catheter steering, flexible electrode deployment, and provides an interface to connect to external devices commonly used in ablation. The proximal handle or hub can include an electrical connection to external devices (e.g., the pulsed RF generator and sensor readers), a control actuator to push and retract the flexible electrode 12 including the hydrogel from or into the distal tip 20, and a steering mechanism (e.g., tension wires) to steer the catheter 10. In the preferred embodiment, the proximal handle or hub provides an interface to the ablation RF generator, a mapping system (involving the electrodes 24), an Electro Physiology (EP) recording system, and irrigation systems connected to the plurality of ports provided in the distal tip 20.

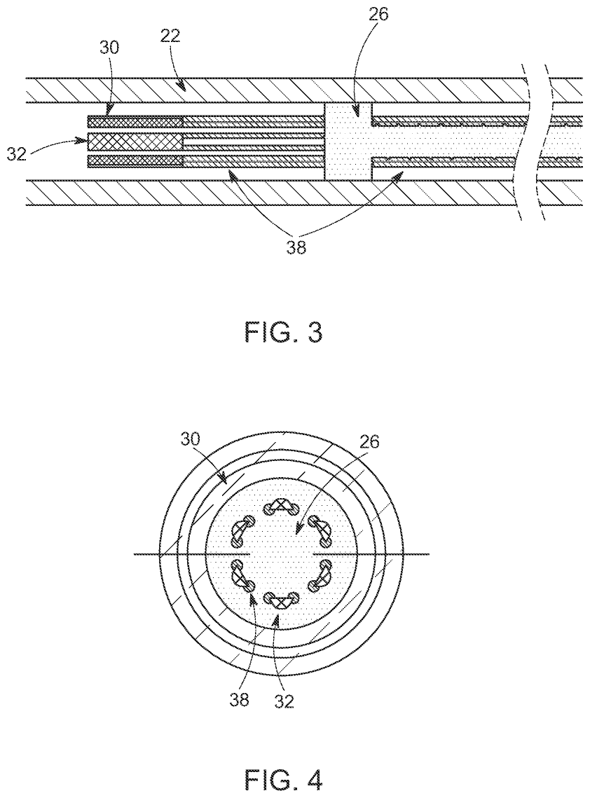

In reference to FIGS. 3 and 4, the body 16 comprises a mechanism to safely deploy and retract the flexible electrode 12, connecting wires 38 to a distal solid electrode 30 and to sensors 32 embedded in a preformed hydrogel (not shown in FIGS. 3 and 4), and the lumen 22 to house these components. The distal solid electrode 30 is in electrical contact with the preformed hydrogel of the flexible electrode 12 once it is deployed and provides electric, radiofrequency power to the flexible electrode 12.

In the preferred embodiment, the catheter body 16 houses conduits for steering, deployment, retraction, irrigation, and appropriate wires for interfacing with sensors. The steering mechanism can include a helical screw, a spring mechanism, or one or more stiff pull wire(s). Deployment and retraction of the hydrogel housed in the lumen 22 of the catheter body 16 may be carried out by pushing and pulling on a plunger base 26. The preformed hydrogel in the inner lumen 22 of the catheter is affixed to the plunger base 26 through mechanical, electrical, or chemical means. The deployment of the hydrogel may be controlled by the control actuator connected to the proximal handle or hub.

The flexible electrode 12 includes a hydrogel with embedded sensors, and/or magnets 32. The flexible electrode 12 can be made of conductive metal wires 38 or solid electrodes 30, one or more different hydrogels, or other flexible materials. The sensors 32 may sense electrical signals, thermal information, hydrogel boundaries, or other relevant information. The flexible electrode 12 is preferably able to be retracted into the tip 20 and resides in the tip 20 during navigation of the catheter 10 to the desired location for ablation.

In use, the hydrogel is already cured, and, in FIG. 2, the hydrogel is deformed. For example, flexible wires, solid electrodes, and sensors are placed in a mold, the uncured hydrogel is poured into the mold, the hydrogel is cured, and the cured piece is retrieved from the mold and attached/connected to a plunger inside a catheter. Alternatively, the hydrogel may be formed around the catheter plunger in the mold as well. The flexible wires and/or solid electrodes are placed into the mold for the uncured hydrogel to cure around. Potential mold shapes include shapes that allow the hydrogel to retract in the lumen 22 and, at the same time, present a large contact area with the target tissue when pressed against it. In each of these example molds, the flexible wire and distal solid electrode are tubular, and centrally located, while the sensors (or plurality of sensors) would be either central (as is illustrated) or on the surface of the hydrogel. However, other shapes of solid electrodes may also be used. Polymerization of the hydrogel can include redox initiation (APS/TEMED, APS/Iron gluconate, Glucose/Glucose oxidase, or any other redox initiation pair), chemical initiation, photoinitiation (Irgacures, Dracurs, or any other photoinitiator), or thermal initiation (AIBN, benzoyl peroxide, potassium persulfate, or any other thermal initiators) among others not listed here. In some embodiments, the polymerization is initiated by the reaction of a reducing agent that is selected from one or more of a hydrocarbon, metal ion, vitamin, enzyme, a ferrous reducing agent, and bioactive agent, with a free radical initiator that is ammonium persulfate, potassium persulfate, or other water-soluble free radical oxidizing initiator. Other methods of curing can involve ultraviolet (UV) light.

The hydrogel is preferably flexible (i.e., soft), deformable (e.g., elastically deformable), and tough (i.e., resistant to fractures). As used herein, “flexible” means having a lower stiffness than the maximum stiffness of the target tissue 14. For example, if the target tissue 14 is myocardial muscle, which has a stiffness that ranges from 20 to 200 kPa, the flexible electrode 12 would be considered flexible if its stiffness is lower than 200 kPa. In some embodiments, “flexible” means at most half as stiff as the maximum stiffness of the target tissue 14. As used herein, “deformable” means having an ultimate elongation corresponding to an average strain of at least 50%. In some embodiments, “deformable” means having an ultimate elongation corresponding to an average strain of at least 100%. The hydrogel material is preferably able to withstand repetitive extension and compression. Flexibility may be achieved by using high molecular hydrophilic polymers such as poly (ethylene glycol) to form the hydrogel. Toughness and fracture/wear resistance may be improved through the incorporation of sacrificial secondary interactions such as hydrogen bonds or ionic bonds. As the hydrogel stretches, the sacrificial secondary interactions are broken and reformed to allow for resistance to the strain with no or little effect on the permanent hydrogel structure or covalent bonds. A hydrogel based on poly (ether urethane diacrylamide) crosslinked with-acryloyl glycinamide, having a stiffness of 59.1±7.5 kPa and an ultimate elongation corresponding to a strain of 111±28%, has been made.

The hydrogel is preferably biostable. To ensure long-term use, the polymers used to form the hydrogel are preferably biostable and resist both hydrolytic and oxidative degradation. These characteristics may be achieved by synthesizing hydrogel macromers without moieties that are susceptible to hydrolysis. Potential hydrogels used include polyethylene glycols, polyurethanes, poly(urethane ureas), poly(vinyl alcohols), polyamides, poly(ether urethane diacrylamide), gelatin, agarose, hyaluronic acid, collagen, fibrin, and any other hydrogel not mentioned. This includes covalently crosslinked, physically crosslinked, dual, and multiple networks. For example, a crosslinker may include N-acryloyl glycinamide.

The hydrogel is preferably biocompatible and non-cytotoxic. The hydrogel and byproducts would not cause harm to the local or systemic tissues. Examples of hydrogel formulations can be found in International application publication no. WO 2021/046441, which is incorporated herein by reference.

The flexible electrode 12 can be connected only to one pole (and the return may be obtained through a separate patch electrode on the patient). The hydrogel is preferably conductive to allow for the propagation of electricity. As used herein, “conductive” means more conductive than the target tissue 14. For example, if the target tissue 14 is myocardial muscle, which has a conductivity that ranges from 0.1 to 6.0 mS/cm, the flexible electrode 12 would be considered conductive if its conductivity is higher than 6.0 mS/cm. In some embodiments, “conductive” means at least two times more conductive than the target tissue 14. Conductivity may be conferred through the addition of conductive elements including conductive polymers such as polyanilines, polypyrroles, and polythiophenes, or other conductive elements such as metallic nano- or micro-particles, graphene, carbon nanotubes, and ionic solutions (NaCl, KCl, or other salts). An ionic hydrogel based on saline solution and having a conductivity of 12.8±1.5 mS/cm has been made. To ensure long-term conductivity, the conductive elements may be directly conjugated to the hydrogel network.

In alternative embodiments, the ablation system may have multiple distal tips that house different preformed shapes of electrodes for specific ablation location needs. The multiple distal tips are connectable to the catheter body 16. The catheter may also deliver bipolar RF ablation. Either or both electrodes of the bipole could be embedded in the hydrogel gel. Indeed, though the hydrogel is conductive, it can prevent a short during RF energy delivery. Alternatively, each electrode may be placed in separate gels separated by a barrier. The barrier is preferably not rigid; for example, it could include hydrogel. The hydrogel may have a sponge-like texture, with an aerated network, or a combination of a continuous structure and a sponge-like structure. The ablation system could be used for ablating nerves or other tissue (liver, kidney). The sponge-like structure may be made either via a gas infusion, sonication mixing, or a chemical reaction that would produce gas bubbles in the hydrogel.

While in FIGS. 1-2, the flexible electrode 12 is retractable inside the lumen 22 of the catheter 10, in other embodiments (not shown), a sheath protecting the flexible electrode 12 may instead be retracted, for example, inside or outside the catheter 10.

In reference to FIGS. 1-4, in use, an appropriate location for ablation is targeted per standard procedure. With the location identified, the catheter 10 is introduced into the heart through an introducer sheath (not shown) and directed to the target location. The catheter 10 is introduced into the body of a patient using standard procedures. That is, an introducer sheath would be placed into the desired vessel, typically a femoral vein, and the catheter is then placed into the sheath. The catheter 10 is then guided by the operator to the cardiac chamber and location desired for ablation using the steering mechanism controllable by the proximal end 18 that bends the distal tip 20. A steering mechanism on the proximal end 18 may also be used to steer the catheter 10 via catheter tip deflection or other means. The user will then deploy the hydrogel out of the distal tip 20 using the actuator on the proximal end 18. For example, the user pushes the actuator on the proximal end 18 that engages with the plunger base 26 in the catheter body 16 to deploy the hydrogel through the distal tip 20. The shape and location of the hydrogel may be visualized using imaging tools such as fluoroscopy, electroanatomical mapping systems, ultrasound and/or other imaging tools commonly used for catheter visualization. The embedded electrode(s) 30 and sensor(s) 32, such as magnetic, electrical, or temperature sensors, in the hydrogel can interface with devices commonly used to gather and visualize signals to provide appropriate therapy. For example, the hydrogel contact with the myocardium can be confirmed with the sensors 32. Once the hydrogel is deployed, controlled radiofrequency energy is provided through the distal electrode 30 to the hydrogel. RF energy is delivered from the standard generator connected to the catheter 10. The embedded electrode(s) 30 and sensor(s) 32 may be used to monitor the lesion geometry, tissue temperature, the impedance of the tissue, conduction velocity, or other parameters to guide ablation. The user can determine when to cease RF energy delivery based on impedance readings from the sensors 32 or other information from the sensors. The user may move the catheter 10 to another location to increase the size of the lesion or to create multiple lesions as required. Once the user determines the procedure is complete, the user can retract the hydrogel back into the distal tip 20 using the plunger actuator on the proximal handle or hub. The hydrogel is retracted by interfacing with the control actuator on the proximal end 18 that engages with the plunger base 26 in the catheter body 16. The entire catheter 10 will then be removed from the body via the introducer sheath.

In addition to the foregoing, the disclosure also contemplates at least the following embodiments:

### Embodiment 1

Embodiment 1 is an apparatus for ablating tissue. The apparatus comprises a catheter and a flexible electrode.

The catheter has a proximal end that is adapted for a user. For example, the proximal end includes a handle or hub. Generally, the handle or hub may control catheter steering, flexible electrode deployment, and/or may provide an interface to connect to external devices commonly used in ablation. The catheter also comprises a body that includes a lumen. The body of the catheter has a distal end that is adapted for introduction in a patient. For example, the lumen may be introduced into an introducer sheath. The introducer sheath may be introduced in the femoral vein. The distal end of the lumen may be extended out of the introducer sheath and guided using a steering mechanism into a cardiac chamber.

The flexible electrode includes a cured hydrogel and is located at the distal end of the catheter body. Preferably, the flexible electrode is at least partially retractable into the lumen of the catheter body, and at least partially deployable out of the lumen of the catheter body. As used herein, “flexible” means that the electrode has a lower stiffness than the maximum stiffness of target tissue (e.g., cardiac tissue) of the patient. Generally, the stiffness of the flexible electrode may range from 20 to 200 kPa. Preferably, the stiffness of the flexible electrode may range from 20 to 100 kPa. In particular, the stiffness of the flexible electrode may have a stiffness of 59.1±7.5 kPa.

### Embodiment 2

Embodiment 2 is an apparatus as described in embodiment 1 wherein the proximal handle or hub includes a control actuator to deploy the flexible electrode out and retract the flexible electrode into the lumen of the catheter body. For example, a user may push the actuator. The actuator may engage with a plunger base in the catheter body to deploy the hydrogel through the distal end. Similarly, the user may pull the actuator. The actuator may engage with the plunger base in the catheter body to retract the hydrogel through the distal end.

### Embodiment 3

Embodiment 3 is an apparatus as described in embodiment 1 wherein the proximal handle or hub includes a control actuator to uncover the flexible electrode from and cover the flexible electrode with a sheath. For example, a user may pull the actuator. The actuator may engage with a sheath around the catheter body and uncover the hydrogel through the distal end. Similarly, the user may push the actuator. The actuator may engage with the sheath around the catheter body and cover the hydrogel through the distal end.

### Embodiment 4

Embodiment 4 is an apparatus as described in any of embodiments 1 to 3 wherein at least a portion, and possibly an entirety, of the cured hydrogel is conductive. As used herein, “conductive” means more conductive than the target tissue (e.g., cardiac tissue) of the patient. Generally, the conductivity of the hydrogel may be higher than 6.0 mS/cm. Preferably, the conductivity of the hydrogel may be higher than 12 mS/com. In particular, the conductivity of the hydrogel may be 12.8±1.5 mS/cm.

### Embodiment 5

Embodiment 5 is an apparatus as described in any of embodiments 1 to 4 wherein the flexible electrode is made by curing hydrogel around a tubular electrode located in a mold. Preferably, the tubular electrode is centrally located in the mold. Preferably, the tubular electrode is made of metal. The tubular may be solid metal or may be made of a metal mesh.

### Embodiment 6

Embodiment 6 is an apparatus as described in any of embodiments 1 to 4 wherein the flexible electrode is configured to deliver bipolar RF ablation. For example, either or both electrodes of the bipole could be embedded in the hydrogel gel. Alternatively, each electrode may be placed in separate gels separated by a barrier.

### Embodiment 7

Embodiment 7 is an apparatus as described in any of embodiments 1 to 6 wherein the flexible electrode has a sponge-like texture, with an aerated network, or a combination of a continuous structure and a sponge-like structure.

### Embodiment 8

Embodiment 8 is an apparatus as described in any of embodiments 1 to 7 wherein the flexible electrode is deformable. As used herein, “deformable” means having an ultimate elongation corresponding to a strain of at least 50%. Preferably, the ultimate elongation may correspond to a strain of at least 100%. In particular, the ultimate elongation may correspond to a strain of 111±28%.

### Embodiment 9

Embodiment 9 is an apparatus as described in any of embodiments 1 to 8 wherein the flexible electrode is connected to an electric generator, preferably a pulsed radiofrequency (RF) generator capable of repeatedly generating voltage wave trains at a frequency between 0.1 and 1 MHz for a duration between 1 millisecond and 100 milliseconds.

### Embodiment 10

Embodiment 10 is an apparatus as described in any of embodiments 1 to 9 specifically adapted for ablating cardiac tissue that is the genesis of the errant and abnormal heartbeats.

### Embodiment 11

Embodiment 11 is a kit comprising an apparatus as described in any of embodiments 1 to 10 and multiple distal tips that house different preformed shapes of electrodes for specific ablation location needs. Each of the multiple distal tips is connectable to the catheter body.

It should be noted that any element of any embodiments 1-11 may further include details related this element that are disclosed in a paragraph or Figure describing the preferred embodiment without including details of other elements that are disclosed in the same or other paragraph or Figure.

Specific embodiments of the invention are shown by way of examples in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

What is claimed is:

1. An apparatus for ablating tissue, comprising:
a catheter having a proximal end adapted for a user and a distal end adapted for introduction in a patient; and
a flexible electrode located at the distal end, the flexible electrode including a cured hydrogel.

2. The apparatus of claim 1, wherein the flexible electrode is retractible in a lumen provided at the distal end.

3. The apparatus of claim 2, wherein the flexible electrode is retractible using a plunger mechanism connected to the proximal end.

4. The apparatus of claim 1, wherein the flexible electrode is coverable by a sheath provided at the distal end.

5. The apparatus of claim 1, wherein at least a portion of the cured hydrogel is conductive.

6. The apparatus of claim 5, wherein the proximal end includes a connector electrically coupled to the flexible electrode and the connector is capable of being connected to an electric generator.

7. The apparatus of claim 5, wherein the cured hydrogel is conductive.

8. The apparatus of claim 1, wherein the flexible electrode is made by curing hydrogel around a tubular electrode located in a mold.

9. The apparatus of claim 8, wherein the flexible electrode is made by curing hydrogel around a tubular solid electrode centrally located in a mold.

10. The apparatus of claim 1, wherein the flexible electrode is configured to deliver bipolar RF ablation.

11. The apparatus of claim 1, wherein the flexible electrode has a sponge-like texture, with an aerated network, or a combination of a continuous structure and a sponge-like structure.

12. The apparatus of claim 1, wherein the flexible electrode is deformable.

13. The apparatus of claim 1, further comprising a steering mechanism controllable with the proximal end.

14. A system for ablating tissue, comprising:
a surgical instrument including:
a catheter having a proximal end adapted for a user and a distal end adapted for introduction in a patient; and
a flexible electrode located at the distal end, the flexible electrode including a cured hydrogel; and
an electric generator connected to the flexible electrode.

15. The system of claim 14, wherein at least a portion of the cured hydrogel is conductive.

16. The system of claim 15, wherein the cured hydrogel is conductive.

17. The system of claim 14, wherein the flexible electrode is deformable.

18. The system of claim 14, further comprising a plurality of distal tips that each includes a corresponding different preformed-shaped electrode, wherein each of the multiple distal tips is connectable at the distal end.

19. The system of claim 18, wherein each of the plurality of distal tips is retractible in a lumen provided at the distal end or coverable by a sheath provided at the distal end.

20. A method of ablating tissue, comprising:
providing an apparatus including:
a catheter having a proximal end adapted for a user and a distal end adapted for introduction in a patient; and
a flexible electrode located at the distal end, the flexible electrode including a cured hydrogel;
introducing the catheter in a patient;
contacting a target tissue of the patient with the flexible electrode; and
ablating the target tissue.

21. The method of claim 20 wherein the target tissue is cardiac tissue.

22. The method of claim 21 wherein the target tissue is the genesis of the errant and abnormal heartbeats.
